(19) United States
(12) Patent Application Publication
(10) Pub. No.: US 2025/0351655 A1
(43) Pub. Date: Nov. 13, 2025

(54) ELECTRONIC DEVICE

(71) Applicant: Innolux Corporation, Miaoli County (TW)

(72) Inventors: Jia-Yuan Chen, Miaoli County (TW); Jui-Jen Yueh, Miaoli County (TW); Tsung-Han Tsai, Miaoli County (TW); Kuan-Feng Lee, Miaoli County (TW)

(73) Assignee: Innolux Corporation, Miaoli County (TW)

(21) Appl. No.: 19/173,828

(22) Filed: Apr. 9, 2025

(30) Foreign Application Priority Data
May 10, 2024 (CN) 202410576451.3

(51) Int. Cl. H10H 29/85; H10F 55/15; H10H 29/14

(52) U.S. Cl. CPC H10H 29/857; H10F 55/15; H10H 29/142

(57) ABSTRACT

An electronic device including an electronic panel and a sensor is disclosed. The electronic panel has a first region, which includes a first element and a second element separated from each other by a first distance, and a second region, which includes a third element and a fourth element separated from each other by a second distance. Each of the first element and the second element has an emission region and a transmission region. The sensor overlaps the first region of the electronic panel, and the sensor is configured to receive a sense signal passing through the transmission region. A first ratio of the second distance to the first distance is ranged from 0.76 to 1.24, and a second ratio of an area of the transmission region to an area of the first element is ranged from 0.52 to 0.96.

### CROSS-REFERENCE TO RELATED APPLICATION

This application claims the priority benefit of China application serial no. 202410576451.3, filed on May 10, 2024. The entirety of the above-mentioned patent application is hereby incorporated by reference herein and made a part of this specification.

### BACKGROUND

### Technical Field

This disclosure relates to an electronic device, in particular to a full-screen electronic device with sensing function.

### Description of Related Art

Existing full-screen electronic devices have sensing functions through the integration of under-screen sensors; however, the display region of a full-screen electronic device is required to have at least one additional transmission region to correspond with the under-screen sensor, and since there is no display element in the transmission region, display defects can easily be noticed by the user when viewing the screen of such full-screen electronic device.

### SUMMARY

Some embodiments of the disclosure are directed to an electronic device, capable of having a relatively good sensing function in the case of a full-screen display.

An electronic device provided according to some embodiments of the disclosure includes an electronic panel and a sensor. The electronic panel has a first region and a second region. The first region includes a first element and a second element, and the first element and the second element are separated from each other by a first distance. The second region includes a third element and a fourth element, and the third element and the fourth element are separated from each other by a second distance. Each of the first element and the second element has an emission region and a transmission region. The sensor is overlapped with the first region of the electronic panel, and the sensor is configured to receive a sensing signal passing through the transmission region. A first ratio of the second distance to the first distance is ranged from 0.76 to 1.24, and a second ratio of an area of the transmission region to an area of the first element is ranged from 0.52 to 0.96.

Based on this, the emission region and the transmission region are disposed in the first region of the electronic device provided by the disclosure, and the disclosure limits a proportion range of the transmission region in the first element of the first region to obtain a relatively good sensing accuracy. This allows the electronic device provided by this disclosure to have a relatively good sensing function in the case of a full-screen display.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate example embodiments of the disclosure and, together with the description, serve to explain the principles of the disclosure.

FIG. 1A is a partial top view of an electronic device according to a first embodiment of the disclosure.

FIG. 1B is an enlarged top view of a region R1 in FIG. 1A.

FIG. 1C is an enlarged top view of a region R2 in FIG. 1A.

FIG. 1D is a schematic cross-sectional view along a section line A1-A1′ in FIG. 1A.

FIG. 1E is a schematic cross-sectional view along a section line A2-A2′ in FIG. 1A.

FIG. 1F is an enlarged top view of a region R3 in FIG. 1A.

FIG. 2 is a graph showing relationship between sensing accuracy of a sensor and a ratio of an area of a transmission region to an area of a first element.

FIG. 3A is a partial top view of an electronic device according to a second embodiment of the disclosure.

FIG. 3B is a schematic cross-sectional view of an embodiment along a section line B-B′ in FIG. 3A.

FIG. 3C is a schematic cross-sectional view of another embodiment along the section line B-B′ in FIG. 3A.

FIG. 3D is a partial top view of an embodiment of a heat dissipation layer in FIG. 3A.

FIG. 3E is a partial top view of an embodiment of arrangement of multiple first pads in FIG. 3A.

FIG. 4 is a partial cross-sectional schematic diagram of an electronic device according to a third embodiment of the disclosure.

FIG. 5 is a partial cross-sectional schematic diagram of an electronic device according to a fourth embodiment of the disclosure.

FIG. 6 is a partial cross-sectional schematic diagram of an electronic device according to a fifth embodiment of the disclosure.

FIG. 7 is a partial cross-sectional schematic diagram of a circuit substrate in an electronic device according to an embodiment of the disclosure.

FIG. 8 is a partial cross-sectional schematic diagram of an electronic device according to a sixth embodiment of the disclosure.

FIG. 9A is a partial cross-sectional schematic diagram of an electronic device according to a seventh embodiment of the disclosure.

FIG. 9B is a partial cross-sectional schematic diagram of an electronic device according to an eighth embodiment of the disclosure.

FIG. 9C is a partial cross-sectional schematic diagram of an electronic device of a nineth embodiment of the disclosure.

FIG. 9D is a partial cross-sectional schematic diagram of an electronic device according to a tenth embodiment of the disclosure.

FIG. 9E is a partial top view of the electronic device according to FIG. 9B.

FIG. 10A is an enlarged top view of an embodiment of a region R4 in FIG. 9C.

FIG. 10B is an enlarged top view of another embodiment of the region R4 in FIG. 9C.

FIG. 10C is an enlarged top view of still another embodiment of the region R4 in FIG. 9C.

### DESCRIPTION OF THE EMBODIMENTS

Reference will now be made in detail to the exemplary embodiments of the disclosure, examples of which are illustrated in the accompanying drawings. Whenever possible, the same reference numerals are used in the drawings and descriptions to refer to the same or similar parts.

This disclosure can be understood by referring to the following detailed description and combined with the accompanying drawings. It should be noted that, in order to make the readers easy to understand and the drawings to be concise, many of the drawings in this disclosure only depict a part of the electronic device, and certain elements in the drawings are not drawn to actual scale. In addition, the number and size of each element in the figure are only for illustration and are not intended to limit the scope of the disclosure.

Certain words are used throughout this disclosure and in the claims to refer to specific elements. Those skilled in the art should understand that electronic device manufacturers may refer to the same element by different names. This disclosure does not intend to distinguish elements that have the same function but different names. In the following description and claims, words such as “include”, “contain”, and “have” are open-ended words, so they should be interpreted as meaning “including but not limited to . . . ”. Therefore, when the terms “include”, “contain” and/or “have” are used in the description of the disclosure, they specify the presence of the corresponding features, regions, steps, operations and/or components, but do not exclude the presence of one or more corresponding features, regions, steps, operations, and/or components.

The directional terms mentioned in this disclosure, such as “up”, “down”, “front”, “back”, “left”, “right”, etc., are only for reference to the directions in the accompanying drawings. Accordingly, the directional terms used are illustrative and not limiting of the disclosure. In the drawings, each figure illustrates the general features of methods, structures, and/or materials used in particular embodiments. However, these drawings should not be interpreted as defining or limiting the scope or nature encompassed by these embodiments. For example, the relative sizes, thicknesses, and locations of layers, regions, and/or structures may be reduced or exaggerated for clarity.

When a corresponding component (such as a layer or region) is referred to as being “on” another component, it can be directly on another component, or other components may be present in between. On the other hand, when a component is referred to as being “directly on” another component, there are no intervening components unless otherwise stated in the specification. In addition, when a component is referred to as being “on” another component, there is an up-and-down relationship between the two in the top direction, and the component may be above or below another component, and this up-and-down relationship depends on the orientation of the device.

The terms “equal to” or “the same”, “substantially” or “roughly” are generally interpreted to mean within 20% of a given value or range, or to mean within 10%, 5%, 3%, 2%, 1% or within 0.5% of a given value or range.

The use of ordinal numbers, such as “first”, “second”, etc., to qualify an element in the specification and claims does not in itself imply or represent any previous ordinal number of the element(s), nor does it represent a sequence of one element and another, or a sequence of manufacturing methods, and the use of such ordinal numbers is only intended to make it possible to clearly distinguish an element with a certain name from another element with the same name. The same words may not be used in the claims and the specification. Accordingly, the first component in the specification may be the second component in the claims.

It should be noted that the following embodiments can be replaced, reorganized, and mixed with features of several different embodiments without departing from the spirit of the disclosure to complete other embodiments. Features in various embodiments may be mixed and matched as long as they do not violate the spirit of the disclosure or conflict with each other.

The term “electrical connection” or “electrically connected” described in this disclosure may refer to either a direct connection or an indirect connection. In the case of direct connection, the terminals of the elements on the two circuits are directly connected or connected to each other by a conductor line, and in the case of indirect connection, there are switches, diodes, capacitors, inductors, other suitable elements, or combinations of the above elements between the terminals of the elements on the two circuits, but the disclosure is not limited thereto.

In this disclosure, the thickness, length, width, and area can be measured using an optical microscope, and the thickness can be measured using cross-sectional images in an electron microscope, but are not limited thereto. In addition, any two values or directions used for comparison may have certain errors. If the first value is equal to the second value, it implies that there may be an error of about 10% between the first value and the second value; if the first direction is perpendicular to the second direction, the angle between the first direction and the second direction may be between 80 degrees and 100 degrees; if the first direction is parallel to the second direction, the angle between the first direction and the second direction may be between 0 degrees and 10 degrees.

The electronic device described in this disclosure can be applied to display devices, light-emitting devices, backlight devices, antenna devices, sensing devices or splicing devices, or to temporary storage substrates that assist electronic units to be placed at specific intervals, but are not limited thereto. The electronic device may be a bendable or flexible electronic device. The display device may be a non-self-luminous display device or a self-luminous display device. The antenna device may be a liquid crystal type antenna device or a non-liquid crystal type antenna device, and the sensing device may be a sensing device that senses capacitance, light, heat energy or ultrasonic waves, but is not limited thereto. Electronic device may include electronic components such as passive elements and active elements, e.g., capacitors, resistors, inductors, diodes, transistors, etc. Diode may include light-emitting diodes or photodiodes. Light-emitting diode (LED) may include, for example, organic light-emitting diode (OLED), sub-millimeter light-emitting diode (mini LED), micro light-emitting diode (micro LED), or quantum dot LED, but not limited thereto. The splicing device may be, for example, a display splicing device or an antenna splicing device, but is not limited thereto. It should be noted that the electronic devices can be arranged in any of the above combinations, but is not limited thereto. In addition, the shape of the electronic device may be a rectangle, a circle, a polygon, a shape with curved edges, or other suitable shapes. The electronic panel may include a display panel, a sensing panel, or an antenna panel, but is not limited thereto.

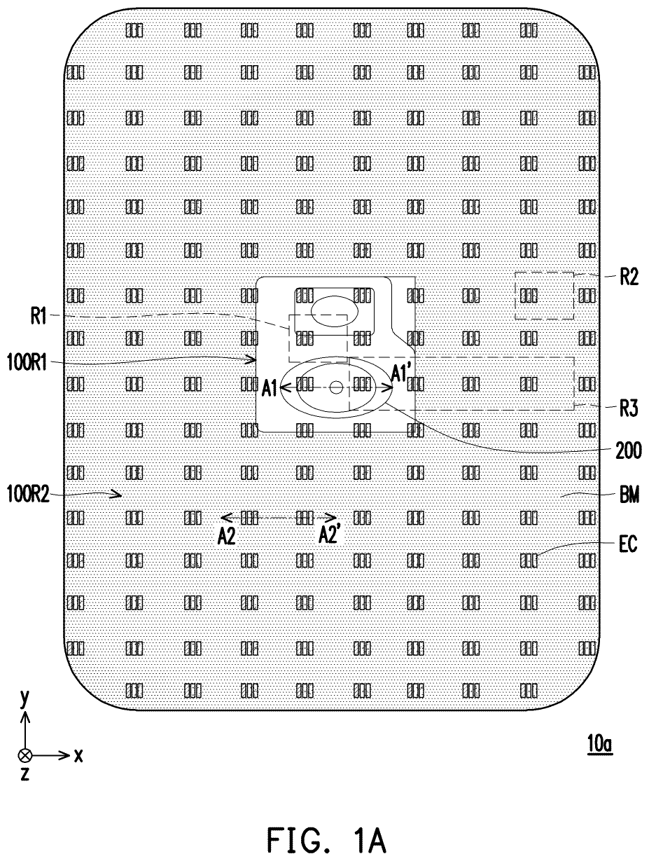

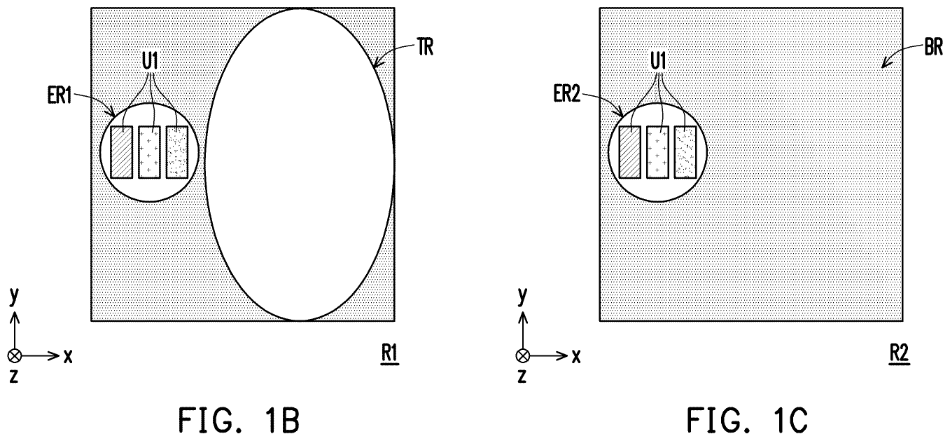

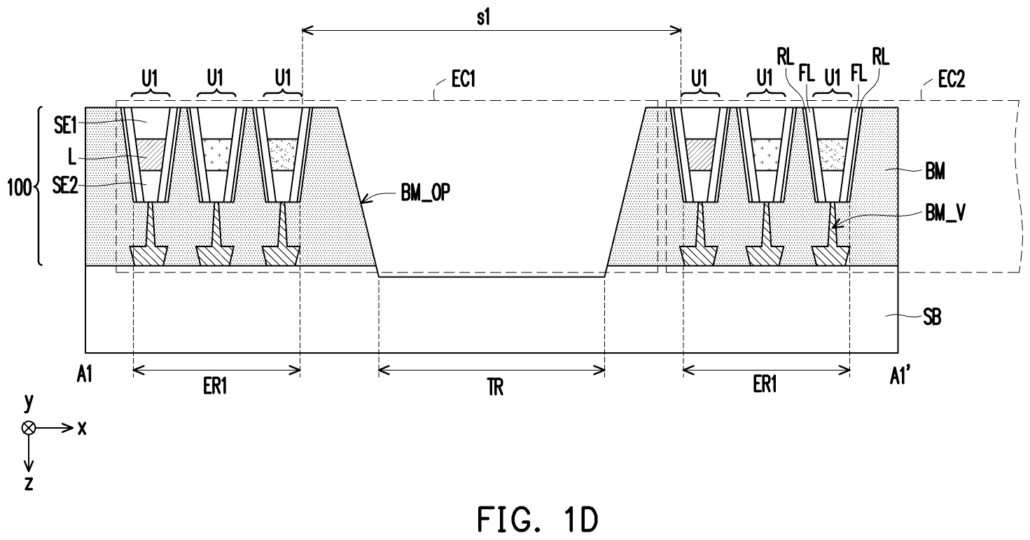

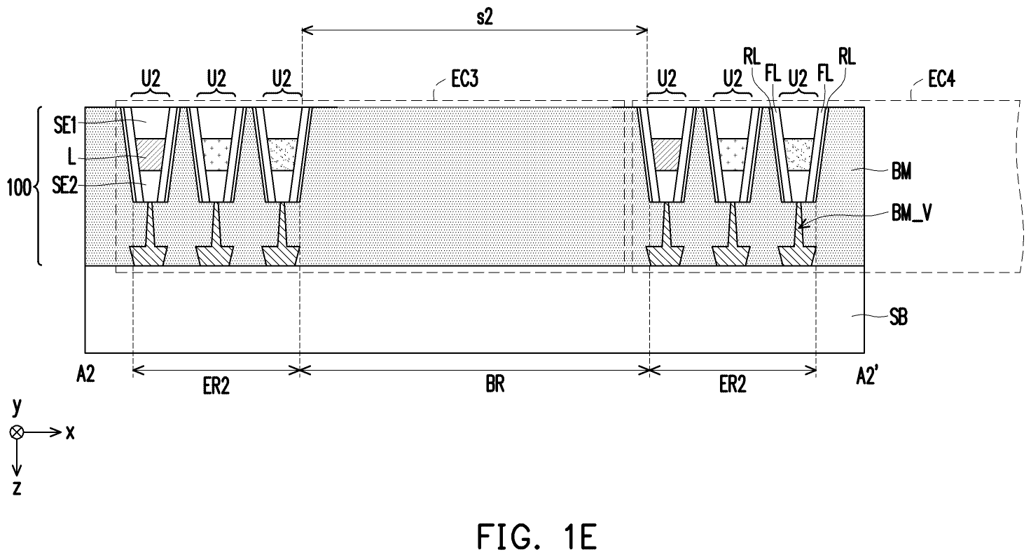

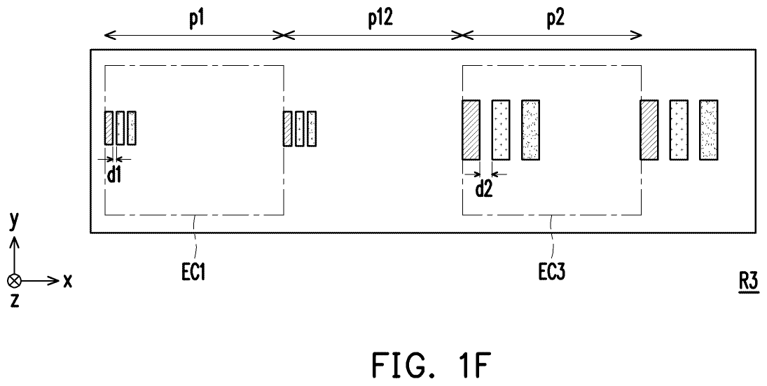

FIG. 1A is a partial top view of an electronic device according to a first embodiment of the disclosure. FIG. 1B is an enlarged top view of a region R1 in FIG. 1A. FIG. 1C is an enlarged top view of a region R2 in FIG. 1A. FIG. 1D is a schematic cross-sectional view along a section line A1-A1′ in FIG. 1A, and FIG. 1E is a schematic cross-sectional view along a section line A2-A2′ in FIG. 1A.

Please refer to FIG. 1A to FIG. 1E at the same time. An electronic device 10a of this embodiment includes a substrate SB, an electronic panel 100, and a sensor 200. That is, in this embodiment, the electronic device 10a is a display device including a sensing function, but the disclosure is not limited thereto.

A material of the substrate SB may be, for example, glass, plastic, or a combination thereof. For example, the material of the substrate SB may include quartz, sapphire, silicon (Si), germanium (Ge), silicon carbide (SiC), gallium nitride (GaN), silicon germanium (SiGe), polymethyl methacrylate (PMMA), polycarbonate (PC), polyimide (PI), polyethylene terephthalate (PET), or other suitable materials or combinations of the above materials, and the disclosure is not limited thereto. In this embodiment, the material of the substrate SB includes glass.

The electronic panel 100 is disposed on the substrate SB, for example. In this embodiment, the electronic panel 100 includes multiple electronic components EC and a blocking layer BM.

The electronic components EC are disposed on the substrate SB, for example. In some embodiments, the electronic components EC may include chips, light-emitting diodes, variable capacitors, variable resistors, varactor diodes, other suitable electronic components, or combinations thereof, and this disclosure is not limited thereto. In this embodiment, at least one of the electronic components EC includes a luminescent element. For example, the electronic component EC may include a diode, an organic light-emitting diode (OLED), inorganic light-emitting diode LED), such as sub-millimeter light-emitting diode (mini LED), micro light-emitting diode (micro LED), quantum dot (QD), quantum dot light-emitting diode (QDLED), fluorescence, phosphor, other suitable materials, or combinations of the above materials, but the disclosure is not limited thereto. In other embodiments, at least one of the electronic components EC may include a communication element. In this embodiment, the electronic component EC is a vertical micro light-emitting diode. In detail, one of the electronic components EC may include, for example, a first semiconductor layer SE1, a second semiconductor layer SE2, and an emission layer L, but the disclosure is not limited thereto. The first semiconductor layer SE1 and the second semiconductor layer SE2 may, for example, each include an N-type doped semiconductor and a P-type doped semiconductor; or each may include a P-type doped semiconductor and an N-type doped semiconductor. Materials of the first semiconductor layer SE1 and the second semiconductor layer SE2 may include, for example, gallium nitride (GaN), indium gallium nitride (InGaN), gallium arsenide (GaAs), aluminum gallium indium phosphide (AlGaInP), or other elements of groups IIIA and VA, or other suitable materials, and the disclosure is not limited thereto. The emission layer L may, for example, have a quantum well (QW), which may be, for example, a single quantum well (SQW), a multiple quantum well (MQW), or other quantum wells. Based on this, electron holes and electrons provided by the first semiconductor layer SE1 and the second semiconductor layer SE2 may be combined in the emission layer L and emit light energy.

In this embodiment, the electronic components EC include a first element EC1, a second element EC2, a third element EC3, and a fourth element EC4, which will be described in detail in the following embodiments.

The blocking layer BM is, for example, disposed on the substrate SB, and is, for example, adjacent to or surrounding the electronic components EC. A material of the blocking layer BM may include, for example, black resin, black photoresist, metal, or a combination thereof, but the disclosure is not limited thereto. In some embodiments, the blocking layer BM includes a via BM_V, and the second semiconductor layer SE2 of the electronic component EC may be electrically connected to a transistor (not shown) and/or a wiring (not shown) disposed on the substrate SB through the via BM_V, but the disclosure is not limited thereto. In this embodiment, the blocking layer BM includes multiple openings BM_OP. In some embodiments, the opening BM_OP may have a circular shape, an elliptical shape, or other suitable shapes in a top view direction z of the electronic device 10a, but the disclosure is not limited thereto. The openings BM_OP can be used to define a transmission region TR of the first element EC1 and the second element EC2, which will be described in detail in the following embodiments.

In some embodiments, the electronic panel 100 may further include a separation layer RL and a filling layer FL.

The separation layer RL is, for example, disposed on the blocking layer BM, and is, for example, disposed adjacent to or surrounding the electronic component EC. In some embodiments, a material of the separation layer RL includes, for example, light-absorbing materials, reflective materials, scattering materials, or combinations thereof, but the disclosure is not limited thereto. The separation layer RL may, for example, reduce the possibility of light emitted by adjacent electronic components EC interfering with each other and/or can, for example, reduce the problem of light leakage from the electronic components EC. In addition, the separation layer RL may also have a heat dissipation function, for example. In some embodiments, the separation layer RL may include a distributed Bragg reflector (DBR), but the disclosure is not limited thereto. The Bragg reflector includes multiple high-refractive-index insulating layers and low-refractive-index insulating layers stacked alternately with each other.

The filling layer FL is, for example, disposed on the blocking layer BM, and is, for example, adjacent to or surrounding the electronic component EC. In some embodiments, the filling layer FL is disposed between the separation layer RL and the electronic component EC, but the disclosure is not limited thereto. The filling layer FL can serve, for example, to secure or protect the electronic component EC. In some embodiments, the filling layer FL may include a transparent material. For example, a material of the filling layer FL may include epoxy resin, acrylic, other suitable materials, or a combination of the above, but the disclosure is not limited thereto.

In this embodiment, the electronic panel 100 has a first region 100R1 and a second region 100R2. The first region 100R1 is, for example, a region where the electronic panel 100 and the sensor 200 overlap in the top view direction z of the electronic device 10a, and the second region 100R2 is, for example, a region where the electronic panel 100 and the sensor 200 do not overlap in the top view direction z of the electronic device 10a. In this embodiment, the second region 100R2 surrounds the first region 100R1, but the disclosure is not limited thereto.

The first region 100R1 of the electronic panel 100 includes, for example, the first element EC1 and the second element EC2. For detailed introduction of the first element EC1 and the second element EC2, please refer to the electronic component EC of the above embodiment. Briefly, the first element EC1 and the second element EC2 in this embodiment can be the above-mentioned vertical micro light-emitting diodes, and therefore will not be repeated in the following. In this embodiment, since the first region 100R1 is a region where the electronic panel 100 and the sensor 200 overlap in the top view direction z of the electronic device 10a, each of the first element EC1 and the second element EC2 has an emission region ER1 and a transmission region TR as shown in FIG. 1B, in which FIG. 1B exemplifies the first element EC1. The emission region ER1 is defined as, for example, an emission area of the first element EC1. In some embodiments, the first element EC1 may have multiple first units U1. Therefore, the emission region ER1 may be defined as, for example, the sum of emission areas of the first units U1. In some embodiments, the first unit U1 may include light-emitting elements that each emit light of the same color or different colors, but the disclosure is not limited thereto. Similarly, the second element EC2 may also have multiple first units U1, and therefore will not be repeated in the following. The transmission region TR is defined as, for example, a region in the electronic panel 100 that allows a sensing signal to be sensed by the sensor 200 to penetrate, which may be defined, for example, by the openings BM_OP of the blocking layer BM.

In some embodiments, an area of the emission region ER1 is less than an area of the transmission region TR, as shown in FIG. 1B. Furthermore, in some embodiments, a ratio of the area of the transmission region TR to an area of the first element EC1 is 0.52 to 0.96, but the disclosure is not limited thereto. In other embodiments, the ratio of the area of the transmission region TR to the area of the first element EC1 is 0.7 to 0.9. In still other embodiments, the ratio of the area of the transmission region TR to the area of the first element EC1 is 0.7 to 0.84. The area of the first element EC1 is defined, for example, as the area of the first region 100R1 divided by a quantity of the first element EC1 in the first region 100R1. For example, when the area of the first region 100R1 is A and the quantity of the first element EC1 in the first region 100R1 is B, the area of the first element EC1 is A/B. In this embodiment, the area of the first element EC1 is the area of the region R1 as shown in FIG. 1B, but the disclosure is not limited thereto. Similarly, the area of the transmission region TR and the area of the second element EC2 may also have the above-mentioned ratio relationship, and therefore will not be repeated in the following.

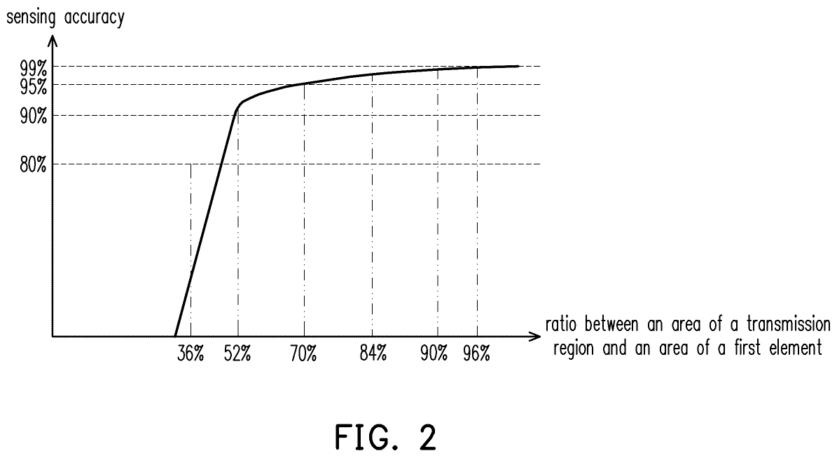

Please refer to FIG. 2. FIG. 2 is a graph showing relationship between sensing accuracy of a sensor 200 and a ratio of an area of a transmission region TR to an area of a first element EC1. The relationship curves are obtained from the results of large-data experiments, in which the number of experiments is more than 100. In this embodiment, by making the ratio of the area of the transmission region TR to the area of the first element EC1 to be 0.52 to 0.96, the sensor 200 may have a sensing accuracy greater than 90%. Based on this, in addition to the display function, the electronic device 10a of this embodiment may also have a relatively good sensing function.

In this embodiment, the first element EC1 and the second element EC2 are spaced apart from each other by a first distance s1. The first distance s1 is defined as, for example, a shortest distance between the first element EC1 and the second element EC2 in a direction perpendicular to the top view direction z of the electronic device 10a. In detail, referring to FIG. 1D, the first element EC1 and the second element EC2 are spaced apart from each other by the first distance s1 in the direction x, in which the first distance s1 is defined as a distance from a first unit U1 in the first element EC1 closest to the second element EC2 to a first unit U1 in the second element EC2 closest to the first element EC1.

The second region 100R2 of the electronic panel 100 includes, for example, a third element EC3 and a fourth element EC4. In some embodiments, the third element EC3 and the fourth element EC4 may each be the same as or similar to the first element EC1 and/or the second element EC2. That is, the third element EC3 and the fourth element EC4 can be, for example, the above-mentioned vertical micro light-emitting diode, which will not be repeated in the following.

In this embodiment, the second region 100R2 is a region where the electronic panel 100 and the sensor 200 do not overlap in the top view direction z of the electronic device 10a. Therefore, each of the third element EC3 and the fourth element EC4 has an emission region ER2 and a blocking region BR as shown in FIG. 1C, in which FIG. 1C exemplifies the third element EC3. The emission region ER2 is defined as, for example, an emission area of the third element EC3. In some embodiments, the third element EC3 has multiple second units U2. Therefore, the emission region ER2 may be defined as, for example, the sum of the emission areas of the second units U2. In some embodiments, the third element EC3 may include light-emitting elements that each emit light of the same color or different colors, but the disclosure is not limited thereto. Similarly, the fourth element EC4 may also have multiple second units U2, and therefore will not be repeated in the following. The blocking region BR is defined as, for example, element and wiring inside the electronic device 10a in the electronic panel 100 that are not intended to be seen by the user, but this disclosure is not limited thereto. In this embodiment, the blocking region BR includes the blocking layer BM. The blocking layer BM is, for example, adjacent to or surrounding the second units U2. In some embodiments, an area of the emission region ER2 is less than an area of the blocking region BR, as shown in FIG. 1C.

In this embodiment, the third element EC3 and the fourth element EC4 are separated from each other by a second distance s2. The second distance s2 is defined as, for example, a shortest distance between the third element EC3 and the fourth element EC4 in a direction perpendicular to the top view direction z of the electronic device 10a. In detail, referring to FIG. 1E, the third element EC3 and the fourth element EC4 are spaced apart from each other by the second distance s2 in the direction x, in which the second distance s2 is defined as a distance from a second unit U2 in the third element EC3 closest to the fourth element EC4 to a second unit U2 in the fourth element EC4 closest to the third element EC3. In this embodiment, a ratio of the second distance s2 to the first distance s1 is 0.76 to 1.24. By making the ratio of the second distance s2 to the first distance s1 to have the above-mentioned ratio range, the electronic device 10a of this embodiment may make display images of the first region 100R1 and the second region 100R2 more consistent in the case of a full-screen display.

In this embodiment, a distance dl between two adjacent first units U1 is less than a distance d2 between two adjacent second units U2. For details, please refer to FIG. 1F. FIG. 1F is an enlarged top view of a region R3 in FIG. 1A. FIG. 1F illustrates that the distance d1 between two adjacent first units U1 in the first element EC1 is less than the distance d2 between two adjacent second units U2 in the third element EC3. In addition, in this embodiment, the size of the first unit U1 is less than the size of the second unit U2. Through the above design, the first element EC1 and the second element EC2 of the first region 100R1 may each include a relatively small-sized emission area ER1, so that the first element EC1 and the second element EC2 may each have a relatively large-sized transmission region TR, whereby the electronic device 10a may have a relatively good sensing function.

Please continue to refer to FIG. 1F. FIG. 1F also shows a pitch p1 between the adjacent first element EC1 and second element EC2 and a pitch p2 between the adjacent third element EC3 and fourth element EC4. The pitch p1 may be defined as, for example, a distance between the adjacent first element EC1 and the second element EC2 at the same relative position, and the pitch p2 may be defined as, for example, a distance between the adjacent third element EC3 and the fourth element EC4 at the same relative position. In this embodiment, the pitch p1 and the pitch p2 may be equal to each other because the size of the first unit U1 and the distance d1 between the two adjacent first units U1 are reduced in equal proportion, but the disclosure is not limited thereto. In addition, it should be noted that the pitch p1 may be, for example, the size of the first element EC1 and/or the second element EC2 in the direction perpendicular to the top view direction z of the electronic device 10a, and the pitch p2 may be, for example, the size of the third element EC3 and/or the fourth element EC4 in the direction perpendicular to the top view direction z of the electronic device 10a. In this embodiment, the pitch p1 is a length of the first element EC1 and the second element EC2 in the direction x and/or a width in the direction y, and the pitch p2 is a length of the third element EC3 and the fourth element EC4 in the direction x and/or a width in the direction y, but the disclosure is not limited thereto. In addition, in this embodiment, a pitch p12 is a distance between the second element EC2 and the third element EC3 in the direction x, which may also be equal to the pitch p1 and the pitch p2, but the disclosure is not limited thereto.

The sensor 200 overlaps with the first region 100R1 of the electronic panel 100, for example. Referring to FIG. 1D, in this embodiment, the sensor 200 overlaps the first region 100R1 of the electronic panel 100 in the top view direction z of the electronic device 10a. For example, the sensor 200 is configured to receive a sensing signal passing through the transmission region TR of the first region 100R1, in which the definition of the transmission region TR may refer to the above embodiment and will not be repeated in the following. In some embodiments, the sensor 200 includes a light sensor that can be used to sense visible light or non-visible light. In this embodiment, the sensor 200 is disposed on a surface of the substrate SB away from the electronic component EC, but the disclosure is not limited thereto. The sensor 200 may include, for example, a visible light sensor, a near-infrared light sensor, an infrared light sensor, a Li-Fi (light fidelity) receiver, or other suitable sensors, and the disclosure is not limited thereto.

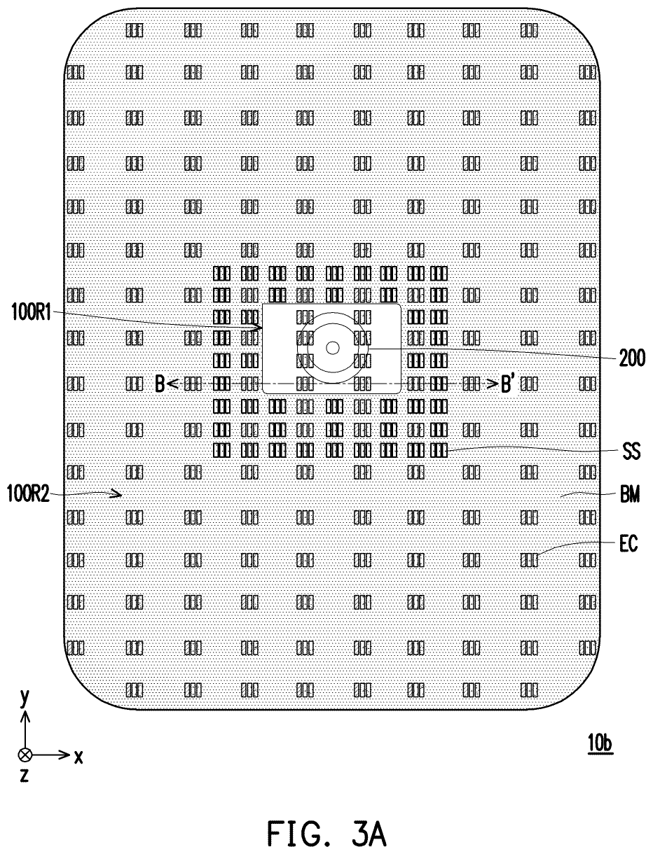

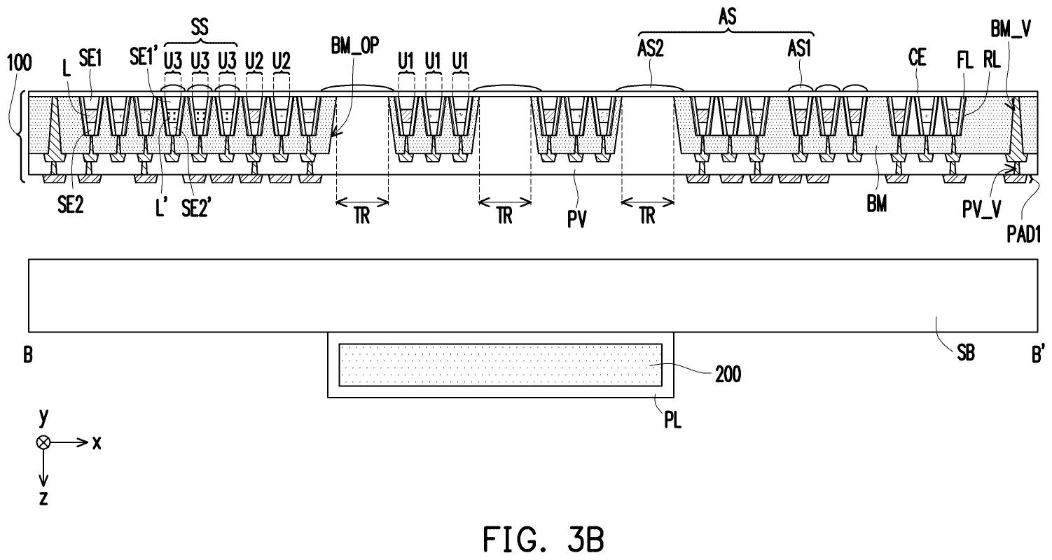

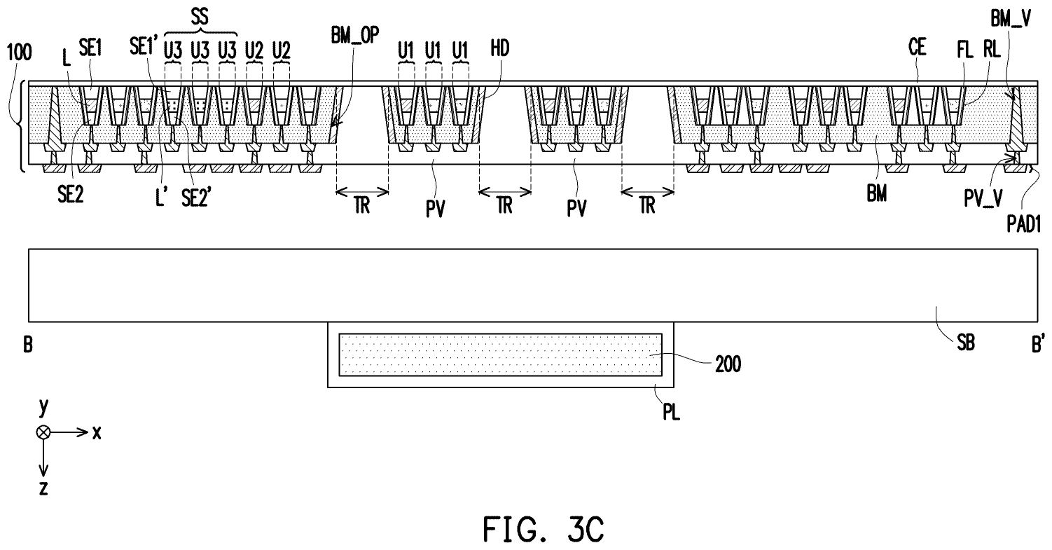

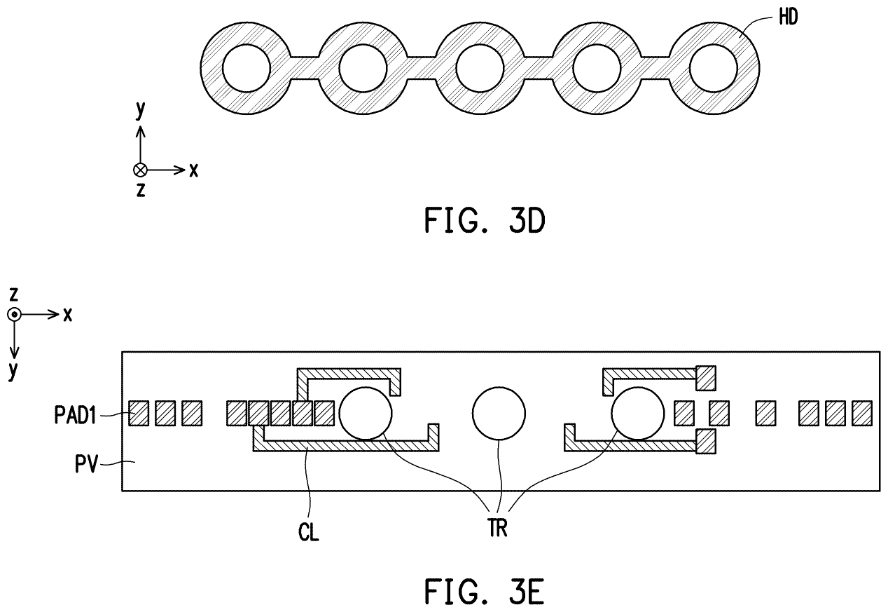

FIG. 3A is a partial top view of an electronic device according to a second embodiment of the disclosure. FIG. 3B is a schematic cross-sectional view of an embodiment along a section line B-B′ in FIG. 3A. FIG. 3C is a schematic cross-sectional view of another embodiment along the section line B-B′ in FIG. 3A, and FIG. 3D is a partial top view of an embodiment of a heat dissipation layer in FIG. 3A. It should be noted that the embodiments of FIG. 3A and FIG. 3B (FIG. 3C) can respectively use the reference numerals and part of the contents of the embodiments of FIG. 1A and FIG. 1D, the same or similar reference numerals are used to represent the same or similar elements, and descriptions of the same technical content are omitted.

Please refer to FIG. 3A and FIG. 3B at the same time. The main difference between an electronic device 10b of this embodiment and the electronic device 10a is that the electronic device 10b also includes a signal source SS.

In detail, in this embodiment, the second region 100R2 of the electronic panel 100 also includes the signal source SS. The signal source SS is configured, for example, to provide a sensing signal, in which a peak wavelength of the sensing signal is, for example, 700 nm to 1400 nm. For example, the signal source SS may include a near-infrared light emitter (NIR emitter). In detail, the signal source SS may include a first semiconductor layer SE1′, a second semiconductor layer SE2′, and an emission layer L′. The emission layer L′ may, for example, include a material suitable for emitting near-infrared light with a peak wavelength of 700 nm to 1400 nm, but the disclosure is not limited thereto. The first semiconductor layer SE1′ and the second semiconductor layer SE2′ can respectively refer to the description of the first semiconductor layer SE1 and the second semiconductor layer SE2 in the above embodiment, and therefore will not be repeated in the following.

It should be noted that although FIG. 3A shows that the signal source SS is arranged in an array in the display region of the electronic device 10b, the disclosure is not limited thereto. In other embodiments, the signal source SS may be disposed in a peripheral region of the electronic device 10b and/or attached to the periphery of the electronic device 10b.

In this embodiment, the electronic device 10b also includes an insulating layer PV, a common electrode CE, and an alignment structure AS.

The insulating layer PV is, for example, disposed between the blocking layer BM and the substrate SB. In this embodiment, the insulating layer PV is disposed in the first region 100R1 and the second region 100R2 of the electronic panel 100, and is filled in the openings BM_OP of the blocking layer BM. The insulating layer PV may, for example, include suitable insulating material to reduce the possibility that the sensing signal to be received by the sensor 200 will be blocked. In this embodiment, the insulating layer PV includes multiple vias PV_V. One of the vias PV_V can be connected to the corresponding via BM_V of the blocking layer BM.

Referring to FIG. 3C, in some embodiments, a heat dissipation layer HD may be further disposed on a side wall of at least one of the openings BM_OP of the blocking layer BM to reduce the heat generated by the electronic component EC. The heat dissipation layer HD may, for example, be electrically connected to the common electrode CE. In addition, referring to FIG. 3D, the heat dissipation layer HD has, for example, a ring shape in the top view direction z of the electronic device 10b, but the disclosure is not limited thereto. In other embodiments, an optical layer (not shown) may be disposed on a side wall of at least one of the openings BM_OP of the blocking layer BM, which may have a function of absorbing and/or reflecting light, but the disclosure is not limited thereto.

Multiple first pads PAD1 are, for example, disposed on a surface of the insulating layer PV close to the substrate SB. The first pad PAD1 may, for example, include a suitable conductive material, but the disclosure is not limited thereto. In some embodiments, each of the first pads PAD1 can be electrically connected to the electronic component EC or the signal source SS through the via BM_V of the corresponding blocking layer BM and the via PV_V connected thereto, but this disclosure is not limited thereto.

Please refer to FIG. 3E, which shows the arrangement of multiple first pads PAD1. In this embodiment, the first pads PAD1 do not overlap with the transmission region TR in the top view direction z of the electronic device 10b. This design may reduce the possibility that the sensing signal to be received by the sensor 200 is affected by the first pads PAD1. In addition, in this embodiment, the wiring CL electrically connected to the corresponding first pad PAD1 also does not overlap with the transmission region TR in the top view direction z of the electronic device 10b, so as to reduce the possibility that the sensing signal received by the sensor 200 will be affected.

The common electrode CE is, for example, disposed on the surface of the insulating layer PV away from the substrate SB, and may, for example, at least partially overlap with the electronic component EC and the signal source SS in the top view direction z of the electronic device 10b. In this embodiment, the common electrode CE is electrically connected to the second semiconductor layer SE2 of the electronic component EC and the second semiconductor layer SE2′ of the signal source SS, and can be electrically connected to the first pad PAD1 through the via BM_V and the via PV_V.

The alignment structure AS is, for example, disposed on the common electrode CE. In this embodiment, the alignment structure AS includes an alignment structure AS1 and an alignment structure AS2. The alignment structure AS1 at least partially overlaps with the signal source SS in the top view direction z of the electronic device 10b, and the alignment structure AS2 at least partially overlaps with the transmission region TR in the top view direction z of the electronic device 10b. In some embodiments, the alignment structure AS includes microlenses. For example, the alignment structure AS can be a biconvex lens, a plano-convex lens, or a concave-convex lens, but the disclosure is not limited thereto. Through the setting of the alignment structure AS, the signal emitted by the signal source SS may form an array optical signal through the alignment structure AS1 and reach a sensing object, and the sensor 200 may receive the aligned optical signal through the alignment structure AS2, which may effectively inhibit the crosstalk phenomenon of the signals from non-corresponding regions or the background noise (the unintended sensing signals), so that the signal-to-noise ratio (SNR) of the signals may be further increased.

It should be noted that in this embodiment, the first unit U1 has a relatively small size (for example, less than 10 microns). Thus, although the first unit U1 at least partially overlaps with the transmission region TR in the top view direction z of the electronic device 10b, the relatively small size may reduce the possibility of affecting the signal sensed by the sensor 200.

In some embodiments, a protective layer PL may be disposed to encapsulate the sensor 200 to protect the sensor 200, but the disclosure is not limited thereto.

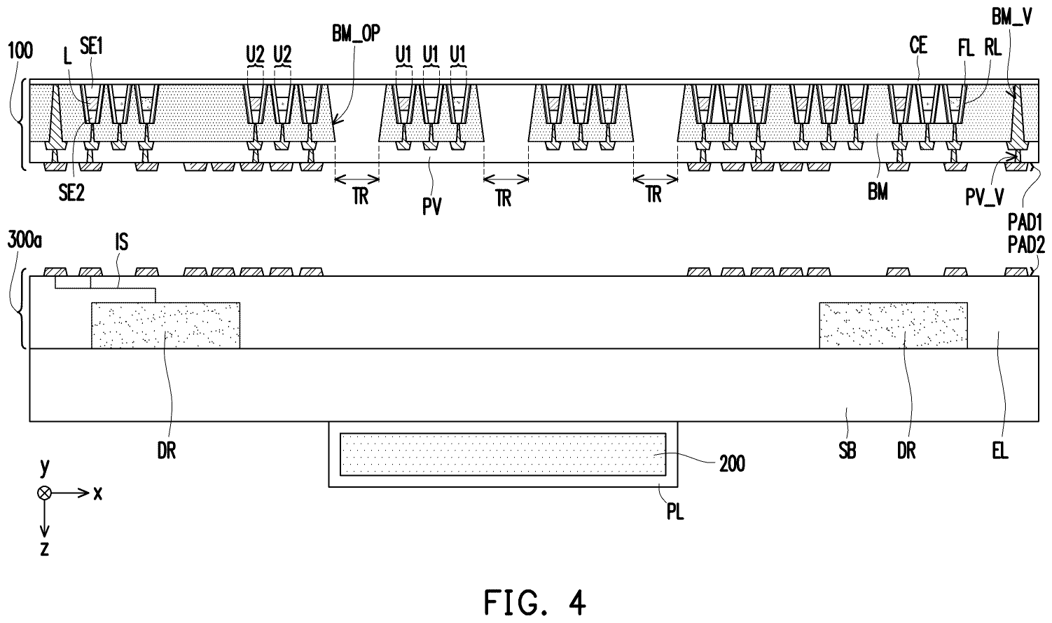

FIG. 4 is a partial cross-sectional schematic diagram of an electronic device according to a third embodiment of the disclosure. It should be noted that the embodiment of FIG. 4 may use the reference numerals and part of the content of the embodiment of FIG. 3B and FIG. 3C, where the same or similar reference numerals are used to represent the same or similar elements, and the description of the same technical content is omitted.

Please refer to FIG. 4. The main difference between an electronic device 10c of this embodiment and the electronic device 10b is that the electronic device 10c also includes a circuit substrate 300a.

In this embodiment, the circuit substrate 300a is disposed on a surface of the substrate SB facing the insulating layer PV, and includes a driver DR, an encapsulation layer EL, and a second pad PAD2.

The driver DR may include, for example, a micro IC. For example, the driver DR may include application-specific integrated circuit chips, analog chips, digital chips, voltage regulator chips, sensor chips and/or memory chips and other suitable semiconductor chips, but the disclosure is not limited thereto. In some embodiments, the driver DR may include an integrated circuit made using silicon wafer as the base material; or may include an integrated circuit made using glass as the base material (glass IC), but the disclosure is not limited thereto. In some embodiments, a chip adhesive film (not shown) may be formed between the driver DR and the surface of the substrate SB facing the insulating layer PV, so that the driver DR can be attached to the substrate SB. A material of the chip adhesive film may include, for example, organic materials, inorganic materials or other suitable adhesive materials, and the disclosure is not limited thereto. In this embodiment, the driver DR is disposed facing upward. In other words, in some embodiments, a connection pad (not shown) may be disposed on a surface of the driver DR away from the substrate SB, but the disclosure is not limited thereto.

In this embodiment, the driver DR does not overlap with the transmission region TR in the top view direction z of the electronic device 10c to reduce the possibility of affecting the signal sensed by the sensor 200.

The encapsulation layer EL is disposed on the driver DR, for example. In this embodiment, the encapsulation layer EL is disposed around the driver DR and includes an interconnector IS, where the driver DR is electrically connected to the interconnector IS. A material of the encapsulation layer EL may be, for example, organic materials or other suitable materials. In this embodiment, the material of the encapsulation layer EL may be epoxy resin, but the disclosure is not limited thereto.

The second pad PAD2 is, for example, disposed on the encapsulation layer EL, and is electrically connected to the driver DR, for example, through the interconnector IS in the encapsulation layer EL. A material of the second pad PAD2 may be, for example, the same as or similar to the material of the first pad PAD1 in the above embodiment, and will not be repeated in the following.

In this embodiment, the first pad PAD1 is coupled to the second pad PAD2. In detail, the first pad PAD1 and the second pad PAD2 can be electrically connected to each other through a redistribution layer (not shown) disposed therebetween, which will be described in detail in the following embodiments.

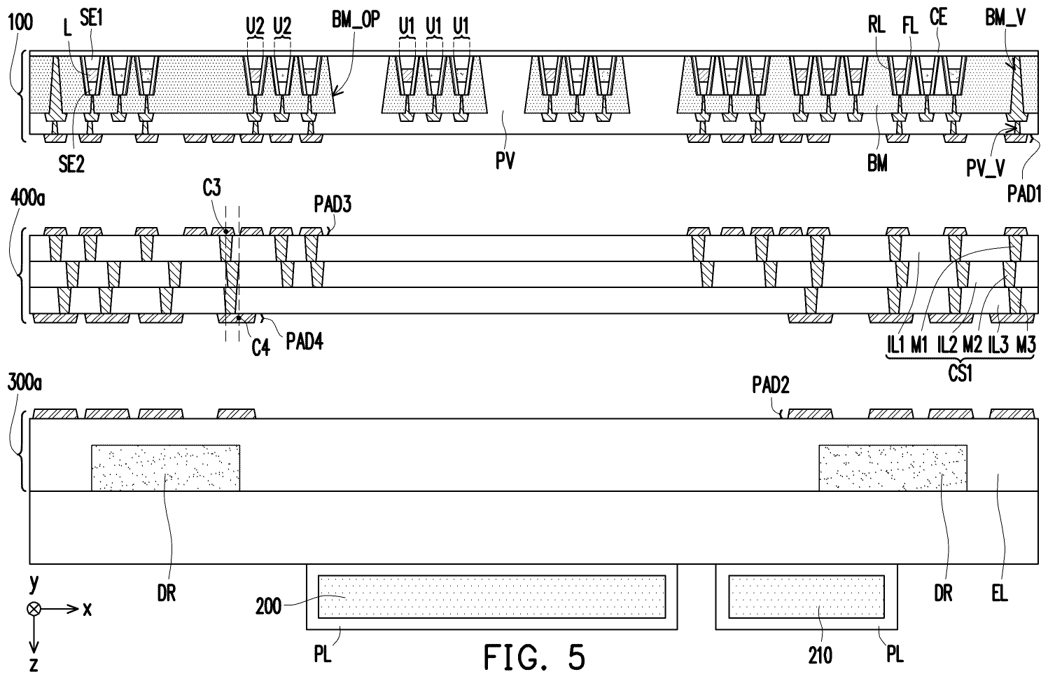

FIG. 5 is a partial cross-sectional schematic diagram of an electronic device according to a fourth embodiment of the disclosure. It should be noted that the embodiment of FIG. 5 may use the reference numerals and part of the content of the embodiment of FIG. 4, where the same or similar reference numerals are used to represent the same or similar elements, and the description of the same technical content is omitted.

Please refer to FIG. 5. The main difference between an electronic device 10d of this embodiment and the electronic device 10c is that the electronic device 10d also includes a redistribution layer 400a and a sensor 210.

The redistribution layer 400a is, for example, disposed between the electronic panel 100 and the circuit substrate 300a. In this embodiment, the redistribution layer 400a includes a connector CS1, multiple third pads PAD3, and multiple fourth pads PAD4.

The connector CS1 may, for example, be used as a distribution layer of the electronic device 10d to provide the required conductive transmission path. For example, as shown in FIG. 5, the connector CS1 may include an insulating layer IL1, a conductive layer M1, an insulating layer IL2, a conductive layer M2, an insulating layer IL3, and a conductive layer M3. Each of the insulating layers IL1, IL2, and IL3 has multiple vias. The corresponding vias of the insulating layer IL1 and the insulating layer IL2 at least partially overlap with the corresponding vias of the insulating layer IL2 and the insulating layer IL3 and are connected to each other. The conductive layer M1, the conductive layer M2, and the conductive layer M3 are each filled in multiple vias of the insulating layer IL1, the insulating layer IL2, and the insulating layer IL3, which are electrically connected to each other through the connected vias to form a conductive transmission path, but the disclosure is not limited thereto.

The third pads PAD3 are, for example, disposed on a surface of the insulating layer IL1 close to the electronic panel 100, and are electrically connected to the conductive layer M1, for example. A material of the third pad PAD3 may be the same as or similar to the material of the first pad PAD1, and therefore will not be repeated in the following. In this embodiment, the third pad PAD3 and the first pad PAD1 are electrically connected. In detail, the third pad PAD3 and the first pad PAD1 may be electrically connected to each other through an interconnector (not shown) or a conductive adhesive layer (not shown) disposed therebetween.

The fourth pads PAD4 are, for example, disposed on a surface of the insulating layer IL3 close to the sensor 200, and are electrically connected to the conductive layer M3, for example. A material of the fourth pad PAD4 may be the same as or similar to the material of the first pad PAD1, and therefore will not be repeated in the following. In this embodiment, the fourth pad PAD4 and the second pad PAD2 are electrically connected. In detail, the fourth pad PAD4 and the second pad PAD2 may be electrically connected to each other through an interconnector (not shown) or a conductive adhesive layer (not shown) disposed therebetween.

Based on the above, in this embodiment, the corresponding third pad PAD3 can be electrically connected to the corresponding fourth pad PAD4 through the connector CS1.

In this embodiment, a center C3 of the third pad PAD3 and a center C4 of the fourth pad PAD4 are offset in the horizontal direction. In detail, referring to the partial cross-sectional view shown in FIG. 5, since the third pad PAD3 and the first pad PAD1 are disposed correspondingly, and the fourth pad PAD4 and the second pad PAD2 are disposed correspondingly, the center C3 of the third pad PAD3 and the center C4 of the fourth pad PAD4 is each offset in the direction x due to the influence of the settings of the first pad PAD1 and the second pad PAD2.

The sensor 210 is, for example, disposed on the surface of the substrate SB away from the electronic component EC, and is adjacent to the sensor 200, for example. In this embodiment, a transmission direction of the signal sensed by the sensor 210 is opposite to a transmission direction of the signal sensed by the sensor 220. Thus, the sensor 210 may overlap with the second region 100R2 of the electronic panel 100 in the top view direction z of the electronic device 10d, but the disclosure is not limited thereto. In this embodiment, the sensor 210 is a sensor using photoplethysmography (PPG). Through the setting of the sensor 210, the electronic device 10d may be used, for example, to detect blood oxygen saturation in the human body. For example, the human heart causes blood vessels to contract and expand with each beat, both of which affect how light is reflected and/or scattered by the vessels. Based on this, when near-infrared light is emitted from a signal source (signal source SS in the above embodiment) to the human body, the sensor 210 can be used to receive the near-infrared light that is reflected and/or scattered by the blood vessels of the human body, and by utilizing a processing circuit (not shown) to read electrical signals (e.g., electrons and/or electron holes) generated by the sensor 210 and calculate the oxygen saturation of the blood vessels in the body. However, this disclosure is not limited thereto. In other embodiments, the electronic device 10d may be applied to biometric identification systems of fingerprints, iris, retina, or other human body organs.

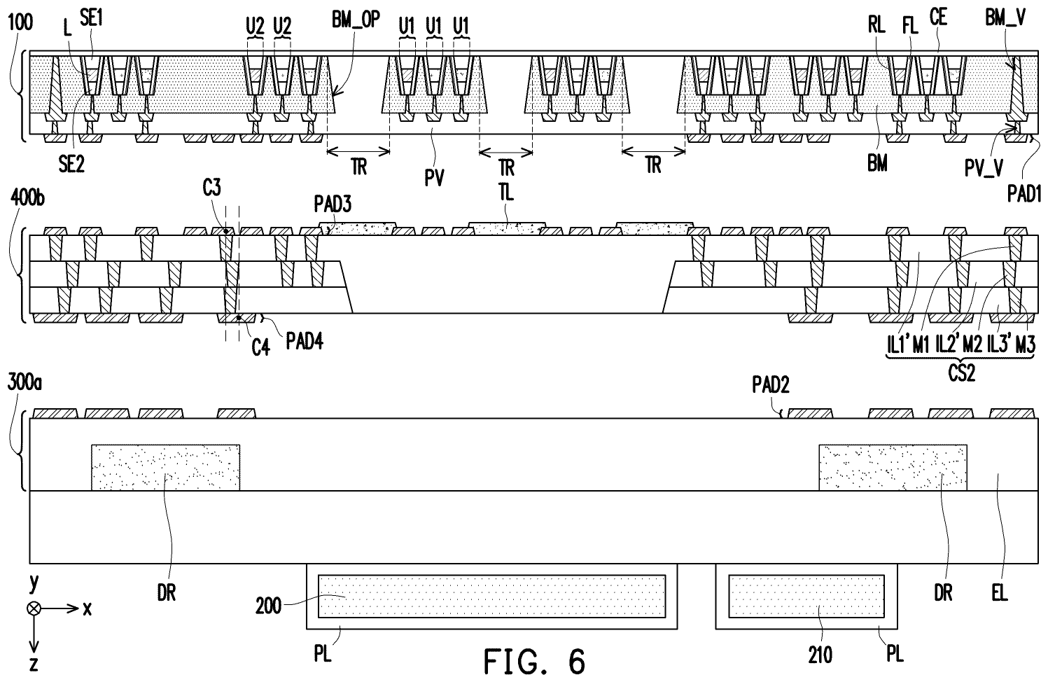

FIG. 6 is a partial cross-sectional schematic diagram of an electronic device according to a fifth embodiment of the disclosure. It should be noted that the embodiment of FIG. 6 may use the reference numerals and part of the content of the embodiment of FIG. 5, where the same or similar reference numerals are used to represent the same or similar elements, and the description of the same technical content is omitted.

Please refer to FIG. 6. The main difference between an electronic device 10e of this embodiment and the electronic device 10d is that the architecture of a redistribution layer 400b in the electronic device 10e is different from the architecture of the redistribution layer 400a.

In detail, the redistribution layer 400b includes, for example, a connector CS2, multiple third pads PAD3, and multiple fourth pads PAD4. The description of the third pad PAD3 and the fourth pad PAD4 may refer to the above embodiments, and therefore will not be repeated in the following.

In this embodiment, the sensor 200 corresponds to an insulating layer in the connector CS2 in the top view direction z of the electronic device 10e. In detail, an insulating layer IL1′, an insulating layer IL2′, and an insulating layer IL3′ of the connector CS2 may have a structure as shown in FIG. 6. The insulating layer IL2′ and the insulating layer IL3′ each have an opening corresponding to the sensor 200 in the top view direction z of the electronic device 10e, and the insulating layer IL1′ is filled in the opening. Based on this, the possibility of the signal received by the sensor 200 being affected by the fact that the insulating layer IL1′, the insulating layer IL2′, and the insulating layer IL3′ have different refractive indices from each other may be reduced.

In addition, in this embodiment, a transparent conductive layer TL may be disposed on a surface of the insulating layer IL1′ close to the electronic panel 100. The transparent conductive layer TL is disposed corresponding to the transmission region TR in the top view direction z of the electronic device 10e, and is electrically connected to the corresponding third pad PAD3. The transparent conductive layer TL can, for example, further provide the conductive transmission path required by the electronic device 10e, and may reduce the possibility of affecting the signal received by the sensor 200.

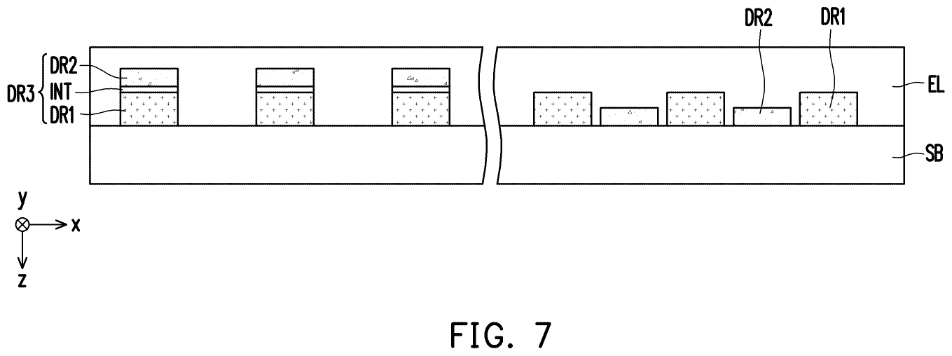

FIG. 7 is a partial cross-sectional schematic diagram of a circuit substrate in an electronic device according to an embodiment of the disclosure. It should be noted that the embodiment of FIG. 7 may use the reference numerals and part of the content of the embodiment of FIG. 4, where the same or similar reference numerals are used to represent the same or similar elements, and the description of the same technical content is omitted.

Please refer to FIG. 7. A circuit substrate 300b of this embodiment shows that the type of the driver DR may include a driver DR1 and/or a driver DR3, and shows the setting relationship between the driver DR1 and a temperature sensor DR2.

The driver DR1 may, for example, include a driver element for controlling the operation of the electronic component EC of the above embodiments, and the temperature sensor DR2 may, for example, include a driver element for sensing the temperature of the electronic component EC of the above embodiments during operation. In some embodiments, the temperature sensor DR2 may include a thermal diode, but the disclosure is not limited thereto.

In some embodiments, the temperature sensor DR2 can be transferred to the same surface of the substrate SB together with the driver DR1, where the temperature sensor DR2 is disposed between adjacent drivers DR1 to be driven by them, but the disclosure is not limited thereto. The density of the temperature sensor DR2 on the substrate SB may be different from the density of the driver DR1 on the substrate SB, but the disclosure is not limited thereto. In other embodiments, the temperature sensor DR2 can be integrated into the driver DR3 through an interposer INT and the driver DR1 using panel level packaging, but the disclosure is not limited thereto. The interposer INT may be, for example, a silicon interposer, a glass interposer, or an organic interposer. For example, multiple micro-bump structures (not shown) may be disposed on a surface of the interposer 200 facing the temperature sensor DR2, so that the temperature sensor DR2 can be bonded to the interposer INT. Although not shown in FIG. 7, in some embodiments, the temperature sensor DR2 can be disposed in an array on a carrier board (not shown), in which the carrier board can be attached to a surface of the substrate SB away from the driver DR1.

In this embodiment, through the setting of the temperature sensor DR2, the temperature sensor DR2 may detect temperature information of each region of the electronic device 10c, and may transmit the information to the driver DR1, so that the driver DR1 can provide the control of the voltage to the electronic component EC on each region accordingly.

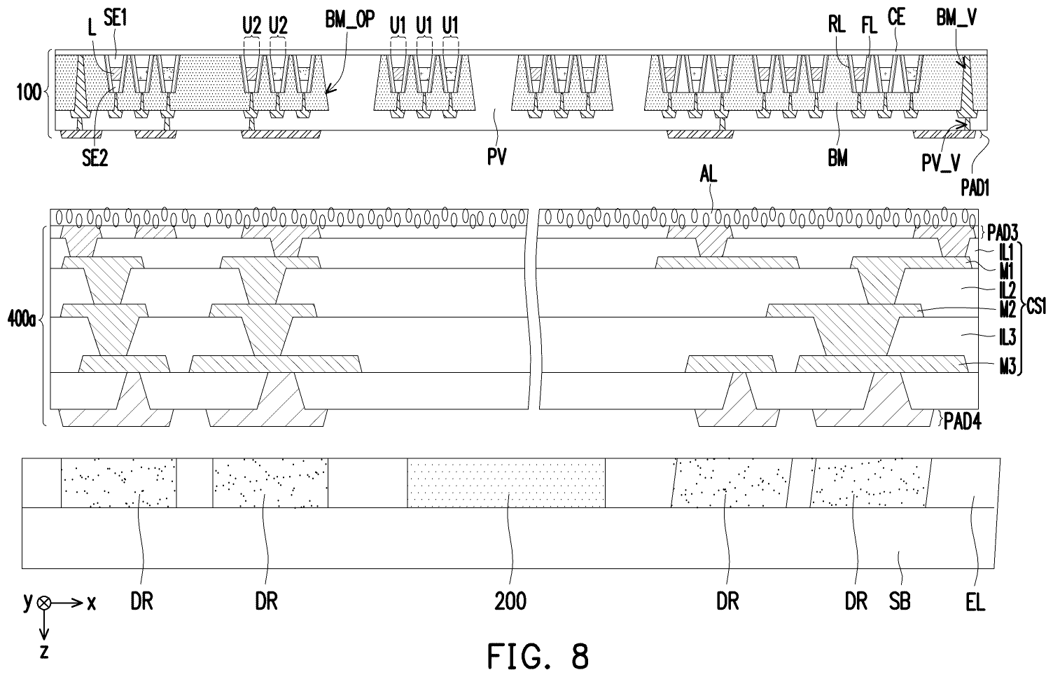

FIG. 8 is a partial cross-sectional schematic diagram of an electronic device according to a sixth embodiment of the disclosure. It should be noted that the embodiment of FIG. 8 may use the reference numerals and part of the content of the embodiment of FIG. 5, where the same or similar reference numerals are used to represent the same or similar elements, and the description of the same technical content is omitted.

Please refer to FIG. 8. The main difference between an electronic device 10f of this embodiment and the electronic device 10d is that the sensor 200 and multiple drivers DR in the electronic device 10f are disposed on the same surface of the substrate SB.

In detail, the sensor 200 may be transferred to a surface of the substrate SB close to the electronic panel 100 together with the drivers DR, for example. In this embodiment, the encapsulation layer EL may surround the sensor 200 and the drivers DR.

In this embodiment, since the driver DR and the sensor 200 are disposed together on the surface of the substrate SB close to the electronic panel 100, the driver DR and the sensor 200 are disposed facing upward. The driver DR can be electrically connected to the conductive layer M3 of the connector CS1 through a bonding pad, but the disclosure is not limited thereto.

In this embodiment, the redistribution layer 400a can be bonded to the electronic panel 100 through an adhesive layer AL. The adhesive layer AL may, for example, include a conductive material. For example, the adhesive layer AL may include anisotropic conductive film (ACF) or other suitable materials, and the disclosure is not limited thereto.

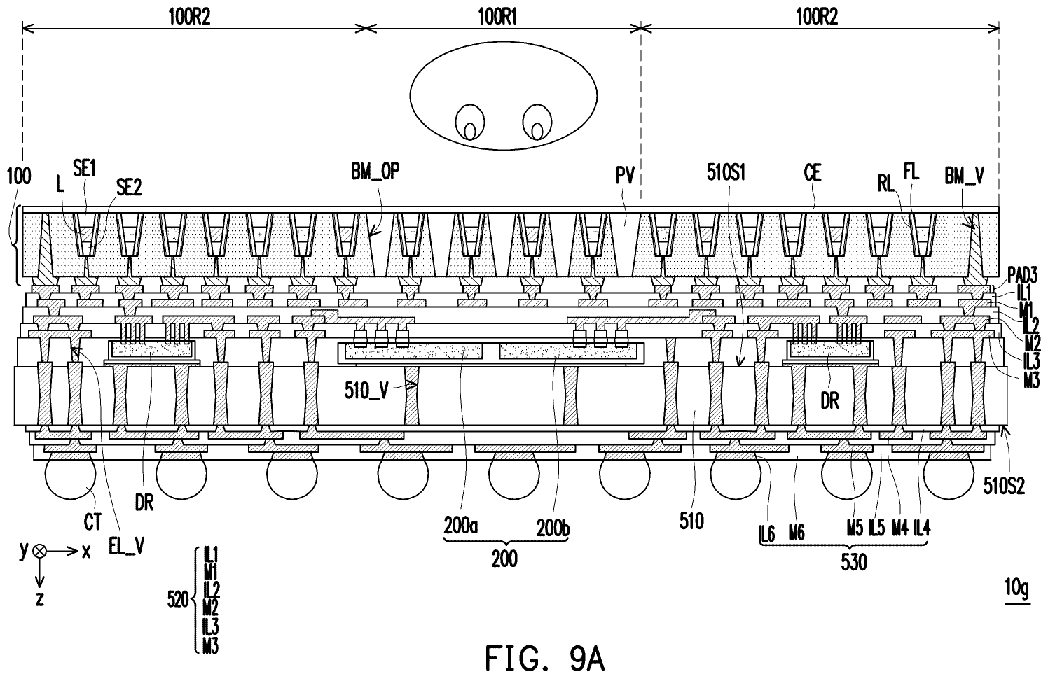

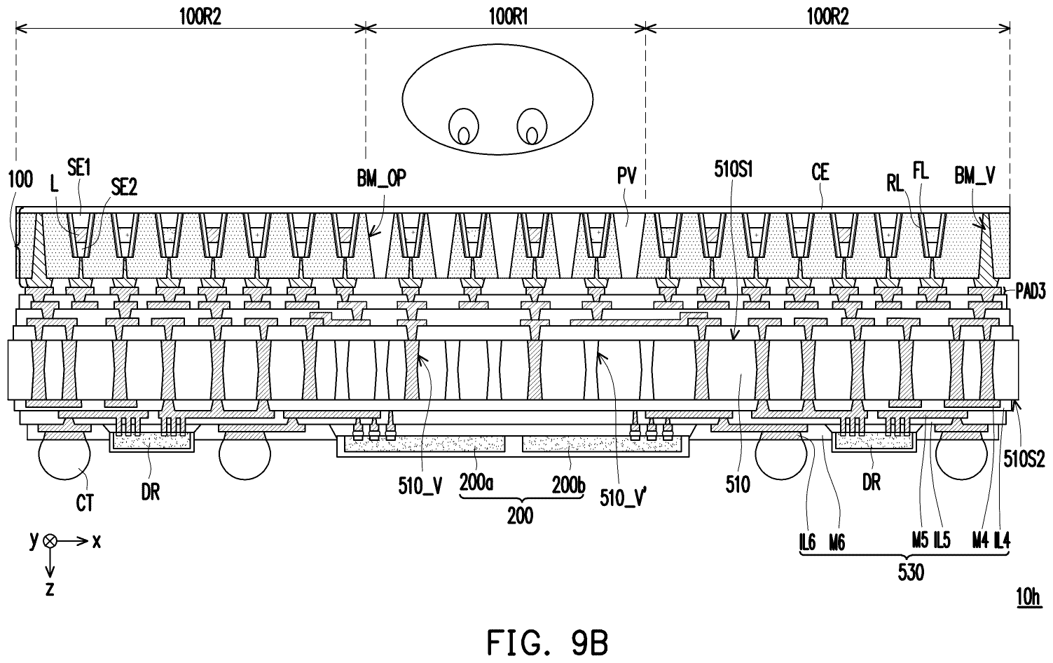

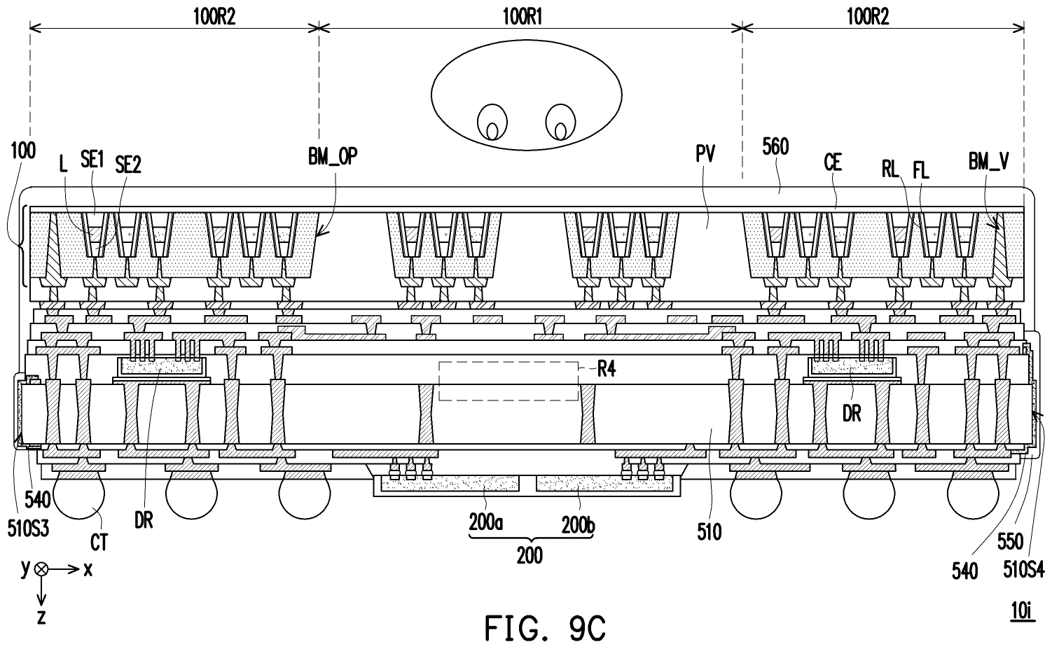

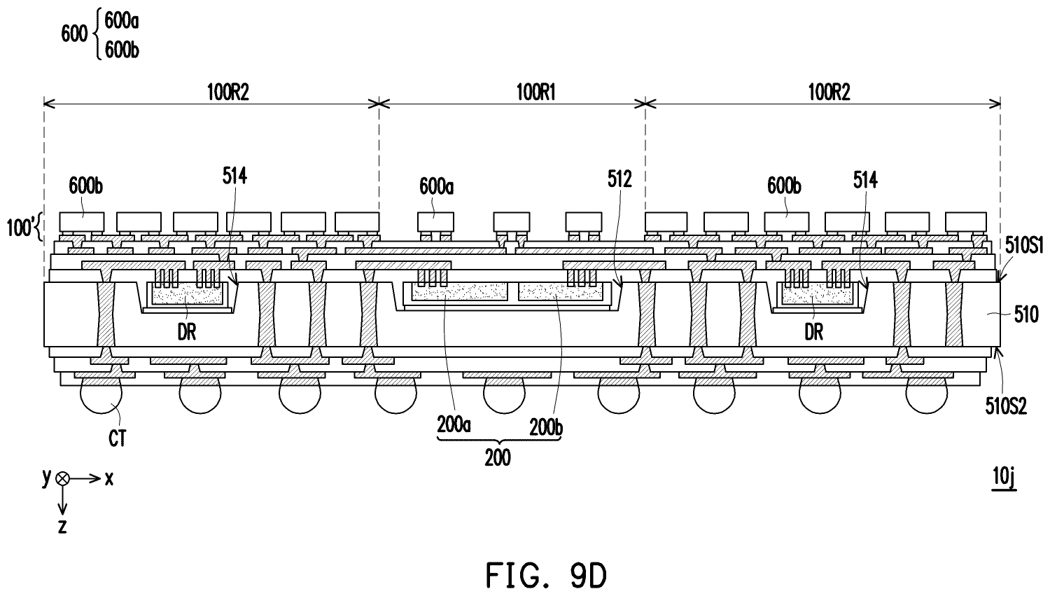

FIG. 9A is a partial cross-sectional schematic diagram of an electronic device according to a seventh embodiment of the disclosure. It should be noted that the embodiment of FIG. 9A may use the reference numerals and part of the content of the embodiment of FIG. 5, where the same or similar reference numerals are used to represent the same or similar elements, and the description of the same technical content is omitted.

Please refer to FIG. 9A. The main difference between an electronic device 10g of this embodiment and the electronic device 10f is that the architecture of a circuit substrate 500 in the electronic device 10g is different from the overall architecture including the circuit substrate 300a, the redistribution layer 400a, and the substrate SB.

In this embodiment, the circuit substrate 500 includes a base layer 510, a first circuit layer 520, and a second circuit layer 530.

The base layer 510 has, for example, a via 510_V. The via 510_V penetrates a first side 510S1 and a second side 510S2 of the base layer 510, and the first side 510S1 is closer to the electronic panel 100 than the second side 510S2. A material of the base layer 510 may be, for example, refer to the description of the substrate SB in the above embodiments, and therefore will not be repeated in the following. In this embodiment, the base layer 510 is made of glass. Based on this, the via 510_V of the base layer 510 may be, for example, a through glass via (TGV). In other embodiments, a material of the base layer 510 includes polymethyl methacrylate (PMMA), polycarbonate (PC), polyimide (PI), polyethylene terephthalate (PET), or other suitable materials or a combination of the above materials.

It should be noted that in this embodiment, the density of the via 510_V overlapping the first region 100R1 in the top view direction z of the electronic device 10g may be less than the density of the via 510_V overlapping the second region 100R2 in the top view direction z of the electronic device 10g. This design may reduce the possibility that the sensing signal to be received by the sensor 200 is affected by the via 510_V.

It should be noted that the second region 100R2 is regarded as a region used for display in the electronic device 10g, and the first region 100R1 is regarded as a region used for both sensing and display in the electronic device 10g, but the disclosure is not limited thereto.

In some embodiments, a crack sensor may be further disposed on the first side 510S1 of the base layer 510. The crack sensor is, for example, disposed in an edge region of the base layer 510 and/or a region where the density of the via 510_V in the base layer 510 changes (such as the junction of the first region 100R1 and the second region 100R2) because the above regions are more prone to cracking.

For example, the first circuit layer 520 is disposed on the first side 510S1 of the base layer 510. In this embodiment, the first circuit layer 520 includes an insulating layer IL1, a conductive layer M1, an insulating layer IL2, a conductive layer M2, an insulating layer IL3, and a conductive layer M3. The way in which the conductive layer M1 to the conductive layer M3 are electrically connected to each other to form a conductive transmission path can be referred to the above embodiment with respect to the connector CS1, and therefore will not be repeated in the following. The first circuit layer 520 is electrically connected to the third pad PAD3 through the conductive layer M1, for example.

The second circuit layer 530 is, for example, disposed on the second side 510S2 of the base layer 510. In this embodiment, the second circuit layer 530 includes an insulating layer IL4, a conductive layer M4, an insulating layer IL5, a conductive layer M5, an insulating layer IL6, and a conductive layer M6. The way in which the conductive layer M4 to the conductive layer M6 are electrically connected to each other to form a conductive transmission path can be referred to the above embodiment with respect to the connector CS1, and therefore will not be repeated in the following. The conductive layer M6 in the second circuit layer 530 can be, for example, a heat dissipation pad, but the disclosure is not limited thereto.

In some embodiments, the conductive layer M6 in the second circuit layer 530 is electrically connected to a conductive terminal CT, thereby being electrically connected to an external electronic component (not shown). The conductive terminal CT may be, for example, a solder ball as shown in FIG. 9A, but the disclosure is not limited thereto. In this embodiment, the first circuit layer 520 and the second circuit layer 530 are electrically connected through the via 510_V in the base layer 510. In detail, the conductive layer M3 in the first circuit layer 520 is electrically connected to the via EL_V of the encapsulation layer EL, for example, and the conductive layer M4 in the second circuit layer 530 is electrically connected to the via 510_V of the base layer 510, for example. Since the corresponding via EL_V of the encapsulation layer EL and the corresponding via 510_V of the base layer 510 at least partially overlap and are electrically connected to each other, the first circuit layer 520 and the second circuit layer 530 may be electrically connected to each other.

In addition, in this embodiment, the circuit substrate 500 is electrically connected to the electronic panel 100 through the third pad PAD3.

In this embodiment, the electronic device 10g also includes a driver DR and a sensor 200. The driver DR is, for example, disposed on the circuit substrate 500, and may be configured, for example, to drive the first element EC1, the second element EC2, the third element EC3, and the fourth element EC4. For example, the driver DR may provide signals to the first element EC1, the second element EC2, the third element EC3, and/or the fourth element EC4 to cause them to emit light or electromagnetic waves, but the disclosure is not limited thereto. The sensor 200 is, for example, disposed on the circuit substrate 500, and may include, for example, a sensor 200a and a sensor 200b. The sensor 200a and the sensor 200b may have the same or different functions, and may be configured to receive the same or different sensing signals, for example. In this embodiment, the driver DR and the sensor 200 are disposed together on the first side 510S1 of the base layer 510. That is, the driver DR and the sensor 200 are disposed facing upward, and each may be electrically connected to the conductive layer M3 of the first circuit layer 520 through the bonding pad, but the disclosure is not limited thereto.

FIG. 9B is a partial cross-sectional schematic diagram of an electronic device according to an eighth embodiment of the disclosure. It should be noted that the embodiment of FIG. 9B may use the reference numerals and part of the content of the embodiment of FIG. 9A, where the same or similar reference numerals are used to represent the same or similar elements, and the description of the same technical content is omitted.

Please refer to FIG. 9B. The main difference between an electronic device 10h of this embodiment and the electronic device 10g is that the driver DR and sensor 200 in the electronic device 10h are disposed together on the second side 510S2 of the base layer 510.

Specifically, the driver DR and the sensor 200 are disposed on the insulating layer IL6 of the second circuit layer 530, and each can be electrically connected to the conductive layer M6 of the second circuit layer 530 through bonding pads CP1, CP2, and CP3, but the disclosure is not limited thereto.

In addition, in this embodiment, the base layer 510 also includes a via 510_V′. For example, the via 510_V′ overlaps with the transmission region TR in the first region 100R1 in the top view direction z of the electronic device 10h. In this embodiment, a material in the via 510_V′ is the same as or similar to the material of the insulating layer PV. This design may reduce the possibility that the sensing signal to be received by the sensor 200 is affected by the base layer 510 having a different refractive index.

It should be noted that, although not shown in FIG. 9B, in other embodiments, the via 510_V′ may include two materials. In detail, a material of an inner layer of the via 510_V′ is, for example, the same as or similar to the material of the insulating layer PV, and a material of an outer layer of the via 510_V′ is, for example, the same or similar to the material of the via 510_V. Through this design, the collimation of the sensing signal to be received by the sensor 200 may be further improved.

FIG. 9C is a partial cross-sectional schematic diagram of an electronic device of a nineth embodiment of the disclosure. It should be noted that the embodiment of FIG. 9C may use the reference numerals and part of the content of the embodiment of FIG. 9B, where the same or similar reference numerals are used to represent the same or similar elements, and the description of the same technical content is omitted.

Please refer to FIG. 9B. The main difference between an electronic device 10i of this embodiment and the electronic device 10h is that the driver DR and the sensor 200 in the electronic device 10i are disposed together on the second side 510S2 of the base layer 510.

For example, a side circuit layer 540 is disposed on the third side 510S3 and the fourth side 510S4 of the base layer 510, and the third side 510S3 and the fourth side 510S4 of the base layer 510 are respectively connected with the first side 510S1 and the second side 510S2. In this embodiment, the side circuit layer 540 is electrically connected to the first circuit layer 520 and the second circuit layer 530 through the second pad PAD2 and the fourth pad PAD4, which may provide additional circuit transmission paths. In addition, in some embodiments, the side circuit layer 540 may further extend from the third side 510S3 and/or the fourth side 510S4 of the base layer 510 to a side surface of the first circuit layer 520 and/or the second circuit layer 530 to communicate with the corresponding conductors in the first circuit layer 520 and/or the second circuit layer 530

A protective layer 550 is, for example, disposed on the third side 510S3 and the fourth side 510S4 of the base layer 510, and covers the side circuit layer 540, for example. In some embodiments, a material of the protective layer 550 may include acrylic resin, polyurethane resin, synthetic rubber resin, or silicone resin. The protective layer 550 may, for example, have relatively high hardness and/or strength to provide, for example, an anti-scratch effect, thereby being used to protect the side circuit layer 540.

An encapsulation layer 560 is disposed on the electronic panel 100, for example. In this embodiment, the encapsulation layer 560 covers the electronic panel 100. The encapsulation layer 560 may be used, for example, to protect the electronic components EC in the electronic panel 100. In this embodiment, the encapsulation layer 560 may be further disposed between the electronic panel 100 and the first circuit layer 520 to protect the first pad PAD1 and the third pad PAD3. In some embodiments, the encapsulation layer 560 may further extend to the side surface of the first circuit layer 520. A material of the encapsulation layer 560 may be, for example, an inorganic material (such as silicon oxide, silicon nitride, silicon oxynitride, or a stacked layer of at least two of the above materials), an organic material (such as polytetrafluoroethylene, polyimide, polyparaxylene, benzocyclobutene, or other suitable materials) or a combination of the above, but the disclosure is not limited thereto.

FIG. 9D is a partial cross-sectional schematic diagram of an electronic device according to a tenth embodiment of the disclosure. It should be noted that the embodiment of FIG. 9D may use the reference numerals and part of the content of the embodiment of FIG. 9A, where the same or similar reference numerals are used to represent the same or similar elements, and the description of the same technical content is omitted.

Please refer to FIG. 9D. The main difference between an electronic device 10j of this embodiment and the electronic device 10g is that (1) an electronic panel 100′ in the electronic device 10j includes multiple communication elements 600; (2) the driver DR and the sensor 200 are embedded in the base layer 510.

The communication element 600 may be suitable for, for example, communication fields, radar/lidar fields, and reconfigurable intelligent surface (RIS) technology, or other suitable fields/technologies, and the disclosure is not limited thereto. In some embodiments, the communication element 600 may include a variable capacitor, a variable resistor, a varactor diode, a phase shifter, an amplifier, an antenna, a biometric sensor, a graphene sensor, other suitable tuning elements, or combinations thereof. For example, the communication element 600 has the function of increasing the operable bandwidth, but the disclosure is not limited thereto.

In this embodiment, the communication element 600 includes a communication element 600a and a communication element 600b. For example, the communication element 600a overlaps with the first region 100R1 in the top view direction z of the electronic device 10j, and the communication element 600b does not overlap with the first region 100R1 in the top view direction z of the electronic device 10j. In this embodiment, the size of the communication element 600a is less than the size of the communication element 600b and/or the density of the communication element 600a is less than the density of the communication element 600b. This design may reduce the possibility that the sensing signal to be received by the sensor 200 is affected by the communication element 600a.

In this embodiment, the base layer 510 has a first recess 512 and a second recess 514, and the sensor 200 and the driver DR are respectively disposed in the first recess 512 and the second recess 514. In some embodiments, the driver DR and the sensor 200 may be embedded together in the first side 510S1 of the base layer 510, but the disclosure is not limited thereto. In other embodiments, the driver DR may be embedded in the first side 510S1 of the base layer 510, and the sensor 200 may be embedded in the second side 510S2 of the base layer 510. At this time, the sensor 200 is disposed facing downward, but the disclosure is not limited thereto.

In some embodiments, at least part of the communication element 600b disposed in the second region 100R2 may also be replaced with a flip-chip light-emitting diode, which allows the second region 100R2 to have both the functions of lighting and communication.

FIG. 9E is a partial top view of the electronic device according to FIG. 9B. It should be noted that the embodiment of FIG. 9E may use the reference numerals and part of the content of the embodiment of FIG. 9B, where the same or similar reference numerals are used to represent the same or similar elements, and the description of the same technical content is omitted.

Referring to FIG. 9E, the electronic device 10h may further include a communication element 600′. In this embodiment, the communication element 600′ is a near field communication (NFC) antenna, but the disclosure is not limited thereto.

The communication element 600′ may be disposed between the electronic panel 100 and the first circuit layer 520, for example. In this embodiment, the wiring CL in the communication element 600′ and cathode wirings of the first unit U1 and the second unit U2 in the electronic panel 100 belong to the same layer. The communication element 600′ can be electrically connected to the common electrode CE through the via BM_V of the blocking layer BM.

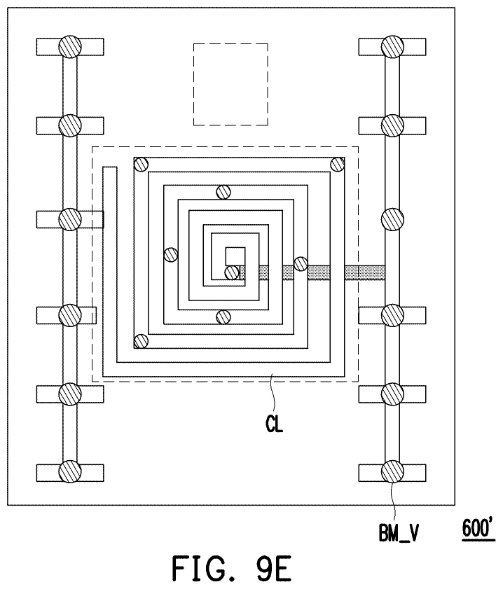

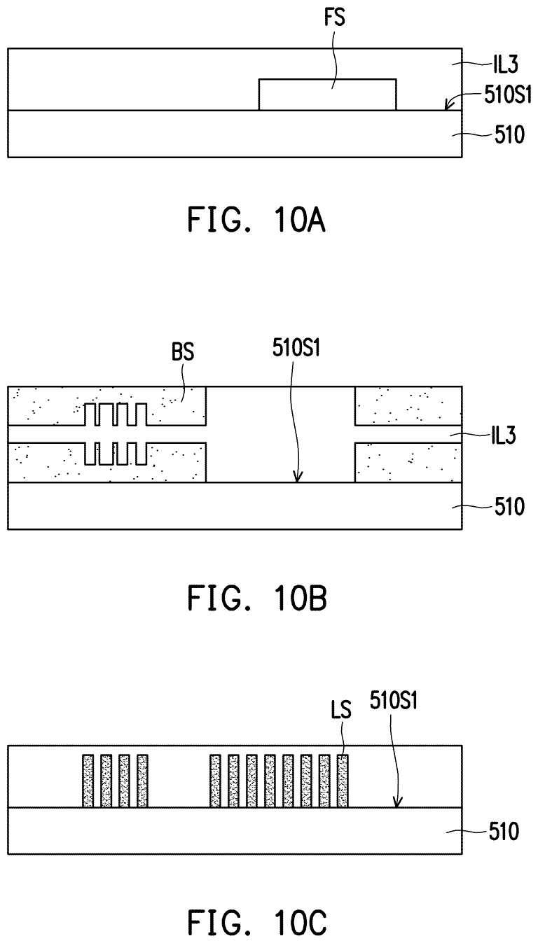

FIG. 10A, FIG. 10B, and FIG. 10C are each an enlarged top view of some embodiments of a region R4 in FIG. 9C. It should be noted that FIG. 10A, FIG. 10B, and FIG. 10C may use the reference numerals and part of the content of the embodiment of FIG. 9C, where the same or similar reference numerals are used to represent the same or similar elements, and the description of the same technical content is omitted.

Referring to FIG. 10A, an electronic device 10i may include a filter structure FS. The filter structure FS is disposed on the base layer 510, for example. In some embodiments, the filter structure FS is disposed on the first side 510S1 of the base layer 510, but the disclosure is not limited thereto. In this embodiment, the filter structure FS at least partially overlaps with the transmission region TR in the top view direction z of the electronic device 10i. Based on this, the sensor 200 can receive the optical signal to be sensed through the filter structure FS, thereby further increasing the signal-to-noise ratio of the optical signal.

Referring to FIG. 10B, the electronic device 10i may include a beam splitter BS. The beam splitter BS is disposed on the base layer 510, for example. In some embodiments, the beam splitter BS is disposed on the first side 510S1 of the base layer 510, but the disclosure is not limited thereto. A material of the beam splitter BS may include, for example, metallic materials or dielectric materials. For example, the material of the beam splitter BS may include aluminum, tin, copper, silver, silicon nitride, or aluminum nitride. In this embodiment, the beam splitter BS at least partially overlaps with the transmission region TR in the top view direction z of the electronic device 10i. Based on this, the beam splitter BS may split the optical signal to facilitate subsequent sensing of the optical signal by the sensor 200.

Referring to FIG. 10C, the electronic device 10i may include a lens structure LS. The lens structure LS is disposed on the base layer 510, for example. In some embodiments, the lens structure LS is disposed on the first side 510S1 of the base layer 510, but the disclosure is not limited thereto. The lens structure LS can be formed, for example, by performing an atomic layer deposition (ALD) process, a physical vapor deposition (PVD) process, or a chemical vapor deposition (CVD) process, but the disclosure is not limited thereto. A material of the lens structure LS may include, for example, metallic materials or dielectric materials. For example, the material of the lens structure LS may include titanium dioxide, aluminum oxide, hafnium oxide, zinc oxide, or silicon nitride. In this embodiment, the lens structure LS at least partially overlaps with the transmission region TR in the top view direction z of the electronic device 10i. Based on this, through the settings of the lens structure LS, the sensor 200 may receive collimated optical signals through the lens structure LS, which may effectively inhibit the crosstalk phenomenon of the signals from non-corresponding regions or the background noise, so that the signal-to-noise ratio of the signals may be further increased.

To sum up, the electronic device provided by some embodiments of the disclosure includes a first region and a second region, where the first region overlapping the sensor is provided with an emission region and a transmission region. Limiting the proportion range of the transmission region in the first region to obtain a relatively good sensing accuracy may enable the electronic device provided by some embodiments of the disclosure to have a relatively good sensing function in the case of a full-screen display. Furthermore, the disclosure may make the first unit in the first region have a relatively small size and/or make the distance between adjacent first units relatively small, and the first region may have a relatively large transmission region, whereby the electronic device provided by some embodiments of the disclosure may have a relatively good sensing function.

In the electronic device provided by other embodiments of the disclosure, by making the distance between adjacent electronic components in the first region and the second region to have a specific ratio range, the electronic device provided by other embodiments of the disclosure may have a relatively good sensing function in the case of a full-screen display.

In the electronic device provided by some embodiments of the disclosure, various structures that may reduce the impact of the sensing signal are formed between the transmission region of the electronic panel and the sensor, thereby further increasing the signal-to-noise ratio of the sensing signal of the sensor.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. An electronic device, comprising:
an electronic panel, having a first region and a second region, wherein the first region comprises a first element and a second element, the first element and the second element are spaced apart from each other by a first distance, the second region comprises a third element and a fourth element, the third element and the fourth element are spaced apart from each other by a second distance, and each of the first element and the second element has an emission region and a transmission region; and
a sensor, overlapped with the first region of the electronic panel, wherein the sensor is configured to receive a sensing signal passing through the transmission region,
wherein a first ratio of the second distance to the first distance is ranged from 0.76 to 1.24, and a second ratio of an area of the transmission region to an area of the first element is ranged from 0.52 to 0.96.

2. The electronic device according to claim 1, wherein the second ratio is ranged from 0.7 to 0.9.

3. The electronic device according to claim 2, wherein the second ratio is ranged from 0.7 to 0.84.

4. The electronic device according to claim 1, wherein an area of the emission region is less than an area of the transmission region.

5. The electronic device according to claim 1, wherein the first element has a plurality of first units, the third element has a plurality of second units, and a distance between two adjacent ones of the first units is less than a distance between two adjacent ones of the second units.

6. The electronic device according to claim 1, wherein the second region further comprises a signal source, the signal source is configured to provide the sensing signal, and a peak wavelength of the sensing signal is ranged from 700 nm to 1400 nm.

7. The electronic device according to claim 1, further comprising a circuit substrate disposed between the electronic panel and the sensor, wherein the electronic panel is electrically connected to the circuit substrate.

8. The electronic device according to claim 7, wherein the electronic panel comprises a first pad, the circuit substrate comprises a second pad, the first pad is bonded on the second pad, the first pad is electrically connected to the first element, and the first pad is not overlapped with the transmission region.

9. The electronic device according to claim 8, further comprising a redistribution layer disposed between the electronic panel and the circuit substrate, wherein the redistribution layer comprises:
a third pad, electrically connected to the first pad;
a fourth pad, electrically connected to the second pad; and
a connector, electrically connected to the third pad and the fourth pad,
wherein a center of the third pad is horizontally shifted from a center of the fourth pad in a cross-sectional view of the electronic device.

10. The electronic device according to claim 1, further comprising a circuit substrate electrically connected to the electronic panel, wherein the circuit substrate comprises:
a base layer;
a first circuit layer, disposed on a first side of the base layer; and
a second circuit layer, disposed on a second side of the base layer,
wherein the first circuit layer and the second circuit layer are electrically connected through a via in the base layer, and the first side is closer to the electronic panel than the second side.

11. The electronic device according to claim 10, wherein the base layer comprises glass.

12. The electronic device according to claim 10, further comprising a driver disposed on the circuit substrate, and configured to drive the first element, the second element, the third element, and the fourth element.

13. The electronic device according to claim 12, wherein the driver and the sensor are disposed on the second side of the base layer.

14. The electronic device according to claim 12, wherein the driver and the sensor are disposed on the first side of the base layer.

15. The electronic device according to claim 12, wherein the driver and the sensor are disposed on the second side and the first side of the base layer respectively.

16. The electronic device according to claim 12, wherein the base layer has a first recess and a second recess, and the sensor and the driver are disposed in the first recess and the second recess respectively.

17. The electronic device according to claim 12, wherein the driver is not overlapped with the transmission region.
